(19) United States
(12) Patent Application Publication
(10) Pub. No.: US 2022/0005572 A1
(43) Pub. Date: Jan. 6, 2022

(54) CANNABINOID AND APPLICATION SUPPORTED OPIOID TAPERING

(71) Applicant: ZYUS Life Sciences US Ltd., Saskatoon (CA)

(72) Inventors: Cedric Francois, Louisville, KY (US); Brent Zettl, Saskatoon (CA)

(21) Appl. No.: 17/479,583

(22) Filed: Sep. 20, 2021

Related U.S. Application Data
(63) Continuation of application No. PCT/IB2020/000221, filed on Mar. 20, 2020.
(60) Provisional application No. 62/821,835, filed on Mar. 21, 2019.

(51) Int. Cl. G16H 20/10; G16H 10/60

(52) U.S. Cl. CPC G16H 20/10; G16H 10/60

(57) ABSTRACT

An application may be used to assist a patient in implementing an opioid taper. Such an application may determine levels of a cannabinoid compound that could help facilitate the patient's taper. This may include receiving information from and/or about the patient, and then using that information to determine a level of a cannabinoid compound that would be appropriate for the patient. Various other functionality, such as providing messages, recommending changes to an opioid prescription, and/or tracking a patient's progress in an opioid taper may also be provided.

### CROSS REFERENCE TO RELATED APPLICATIONS

This is related to, and claims the benefit of, U.S. provisional patent application 62/821,835, titled Cannabinoid and Application Supported Opioid Tapering, filed on Mar. 21, 2019. That application is hereby incorporated by reference herein in its entirety.

### BACKGROUND

Patients who have been prescribed opioids may wish to taper their usage for a variety of reasons, ranging from preparation for future medical procedures (e.g., surgery) to a preference for avoiding medications that have been associated with addiction and dependency. However, opioid tapering can be difficult, and there is a need for improved technology that can assist patients in successfully tapering opioid use.

### SUMMARY

According to a first aspect, some embodiments may provide a method of using an application to implement an opioid taper for a patient. In some embodiments, such a method may comprise the application repeatedly performing a set of tapering steps. In some such embodiments, such a set of tapering steps may include evaluating progress of the patient on the opioid taper. In some such embodiments, such a set of tapering steps may comprise, based on the progress of the patient on the opioid taper, determining a recommended level for administration of a cannabinoid compound. Additionally, in some embodiments, a method of using an application to implement an opioid taper for a patient may comprise administering the cannabinoid compound to the patient at the recommended level.

According to a second aspect, in some embodiments such as described in the context of the first aspect, a method of using an application to implement an opioid taper for a patient may comprise, during an acclimatization period, providing one or more messages to the patient regarding continuing his or her opioid usage at a pre-taper level, and providing one or more messages to the patient regarding use of the cannabinoid compound. Additionally, in some such embodiments, such methods may comprise, after the acclimatization period but before performing the set of tapering steps, the application evaluating appropriateness of the cannabinoid compound for the patient. Additionally, in some such embodiments the performance of the set of tapering steps may be initiated based on the determination that the cannabinoid compound is appropriate for the patient.

According to a third aspect, some embodiments may provide a method such as described in the context of the second aspect may be provided in which the application used in that method may be configured to, during and/or at the conclusion of the acclimatization period, gather cannabinoid side effect information for the patient. In some such methods, the application may be configured to evaluate appropriateness of the cannabinoid compound for the patient based on the gathered cannabinoid side effect information.

According to a fourth aspect, in some embodiments such as described in the context of any of the first through third aspects, a method may be provided in which the set of tapering steps may further comprise, based on the progress of the patient on the opioid taper, determining whether to make one or more recommendations. In some such embodiments, the recommendations may be selected from the group consisting of modifying the opioid taper and changing the patient's opioid prescription.

According to a fifth aspect, in some embodiments such as described in the context of any of the first through fourth aspects, a method may be provided in which the set of tapering steps may comprise gathering information regarding the patient's opioid taper. In some such methods, the gathered information may comprise actual opioid usage and pain assessment data. In some such methods, the application may be configured to evaluate the progress of the patient on the opioid taper based on the gathered information.

According to a sixth aspect, in some embodiments such as described in the context of any of the first through fifth aspects, a method may be provided in which the method comprises, after repeating the set of opioid tapering steps two or more times, ceasing the opioid taper with the patient on an opioid maintenance dosage. In some such methods, the opioid maintenance dosage may be a lower dosage than the patient's pre-taper opioid usage level.

According to a seventh aspect, in some embodiments such as described in the context of any of the first through sixth aspects, a method may be provided in which the patient has a computing device which executes code for the application, the patient's computing device may be configured by the code for the application to communicate with a remote system over a network, and the evaluating and determining steps of the method may be performed by a server at the remote system.

According to an eighth aspect, some embodiments may provide computer program products comprising non-transitory computer readable media having computer executable instructions stored thereon for implementing an opioid taper, and/or methods of treatment comprising prescribing an application and a cannabinoid compound to a patient that correspond to embodiments as described in the context of any of the first through seventh aspects.

According to a ninth aspect, some embodiments may provide a method of using an application in combination with a drug to implement an opioid taper for a patient. In some embodiments, such a method may comprise the application repeatedly preforming a set of tapering steps. In some embodiments, such a set of tapering steps may comprise receiving information regarding progress of the patient on the opioid taper and using that information in evaluating progress of the patient on the opioid taper. In some embodiments, such a method may comprise providing one or more messages to the patient and information to the patient's physician based on the progress of the patient on the opioid taper, wherein at least one of the one or more messages provided to the patient comprises a recommended level for administration of the drug.

According to a tenth aspect, in some embodiments such as described in the context of the ninth aspect, a method may be provided in which the method further comprises administering the drug to the patient at the recommended level.

According to an eleventh aspect, in some embodiments such as described in the context of any of the ninth through tenth aspects, the drug may be a cannabinoid compound.

According to a twelfth aspect, in some embodiments such as described in the context of any of the ninth through eleventh aspects, the set of tapering steps may comprise determining the recommended level for administration of the drug.

According to a thirteenth aspect, in some embodiments such as described in the context of the twelfth aspect, the recommended level may comprise a level prescribed by the patient's physician. In some such embodiments, the one or more messages provided to the patient may comprise a reminder to take the drug as prescribed by the patient's physician.

According to a fourteenth aspect, in some embodiments such as described in the context of any of the ninth through thirteenth aspects, the opioid taper may comprise prescriptions by the patient's physician for an opioid and for the drug. In some such embodiments, the set of tapering steps may comprise, based on evaluation of progress of the patient on the opioid taper, determining whether to recommend modification to the prescriptions by the patient's physician for the opioid and/or for the drug. In some such embodiments, the set of tapering steps may comprise, when a determination is made to recommend modification to the prescription by the patient's physician for the opioid and/or for the drug, sending messages to the patient and the patient's physician recommending the modification. In some such embodiments, the set of tapering steps may comprise, when the patient's physician accepts a recommendation to modify the prescription for the drug, the recommended level comprises a modified level for the drug prescribed by the patient's physician.

According to a fifteenth aspect, in some embodiments such as described in the context of the fourteenth aspect, the application may be configured to recommend modifying the prescription by the patient's physician for the opioid by changing the prescription for the opioid to a smaller unit size without changing formulation.

According to a sixteenth aspect, in some embodiments such as described in the context of any of the ninth through fifteenth aspects, a method may be provided that comprises, during an acclimatization period, the application providing one or more messages to the patient regarding continuing his or her opioid usage at a pre-taper level. In some such embodiments, the method may comprise, during an acclimatization period, the application providing one or more messages to the patient regarding use of the drug. In some such embodiments, the method may comprise, after the acclimatization period but before performing the set of tapering steps, the application evaluating appropriateness of the drug for the patient. In some such embodiments, performance for the set of tapering steps may be initiated based on determining that the drug is appropriate for the patient.

According to a seventeenth aspect, in some embodiments such as described in the context of the sixteenth aspect, the application may be configured to, during and/or at the conclusion of the acclimatization period, gather drug side effect information for the patient. In some such embodiments, the application may be configured to evaluate the appropriateness of the drug for the patient based on the gathered drug side effect information.

According to an eighteenth aspect, in some embodiments such as described in the context of any of the ninth through seventeenth aspects, the information regarding progress of the patient on the opioid taper may comprise outcome information regarding the opioid taper, actual opioid usage, and pain assessment data.

According to a nineteenth aspect, in some embodiments such as described in the context of any of the ninth through eighteenth aspects, a provided method may comprise, after repeating the set of opioid tapering steps two or more times, ceasing the opioid taper with the patient on an opioid maintenance dosage. In some such embodiments, the opioid maintenance dosage may be a lower dosage than the patient's pre-taper opioid usage level.

According to a twentieth aspect, in some embodiments such as described in the context of any of the ninth through nineteenth aspects, the patient may have a computing device which executes code for the application. In some such embodiments, the patient's computing device may be configured by the code for the application to communicate with a remote system over a network. In some such embodiments, evaluating progress of the patient on the opioid taper may be performed by a server at the remote system.

According to a twenty first aspect, in some embodiments such as described in the context of any of the ninth through twentieth aspects, providing information to the patient's physician based on the progress of the patient on the opioid taper may comprise providing the patient's physician an interface indicating amount of medication being taken by the patient on the opioid taper, and changes in status of the patient on the opioid taper.

According to a twenty second aspect, some embodiments may provide a computer readable medium having stored thereon code which, when executed, causes a computer to provide an application as described in the context of any of the ninth through twenty first aspects.

According to a twenty third aspect, some embodiments may provide a system comprising a computer configured to provide an application as described in the context of any of the ninth through twenty first aspects.

Other aspects and embodiments of the disclosed technology are also possible and will be immediately apparent to, and could be implemented without undue experimentation by, one of ordinary skill in the art in light of this disclosure. Accordingly, the above description of various aspects and embodiments should be understood as being illustrative only, and should not be treated as limiting.

### BRIEF DESCRIPTION OF THE DRAWINGS

FIG. 1 is an illustration of an exemplary opioid tapering process.

FIG. 2 is an illustration of an exemplary environment where aspects of the disclosed technology could be deployed.

FIGS. 3A-3C depict exemplary interfaces that may be provided in some embodiments.

### GLOSSARY

Certain terms used in the present application are collected here for convenience. General or specific features of the description of terms in this glossary may be applied in or to any aspect, embodiment, context, description, or claim in which such term is used.

The term “knowledge base” refers to a collection of information that a system may use for performing one or more processes or methods. The term “knowledge base” is not intended to imply any particular structure. The information in a knowledge base may be embodied as a set of rules, as entries in a table, or in any other suitable way. Knowledge in a knowledge base may embody the knowledge of practitioners in medicine or a particular health care discipline.

The term “status” refers to one of a set of states for a patient.

The term “drug” refers to a substance that is intended for use in the diagnosis, cure, mitigation, treatment or prevention of disease, or is intended to affect the structure or any function of the body, including but not limited to substances that have not yet received regulatory approval or been recognized by an official pharmacopia or formulary.

The term “monitoring device” refers to a device that can be used for detecting or measuring one or more physiological variables, environmental variables, or behavioral variables relevant to a patient. Many monitoring devices of interest herein are devices that can be used by a patient in his or her daily life, i.e., while the patient is not under care in a health care facility. Such monitoring devices may be referred to as “personal monitoring devices”. Unless otherwise indicated a monitoring device may be a personal monitoring device. Monitoring devices include: body weight scale, pulse oximeter, blood pressure monitor, thermometer, activity tracking device, heart rate monitor, blood glucose measuring device (glucometer), stethoscope, medication dispensers that in some way monitor medication usage, wearable devices, and any other device capable of obtaining physiological data, environmental data, or behavioral data relating to a patient in his or her daily life.

The term “connected monitoring device” refers to a monitoring device that is or can be connected to a communication network or to a computer that is or can be connected to a communication network such that data collected by the device can be obtained by a system of the present invention without a user entering the data manually. A computer, e.g., a mobile device such as a wearable device, equipped with or in communication with one or more appropriate sensors may serve as a connected monitoring device. Connected monitoring devices also include implanted, indwelling, or swallowed devices that are capable of wirelessly transmitting physiological data to a computer. Connected monitoring devices may be located in (as an integral part) or on of a mobile device or mobile device case. A connected monitoring device is typically a personal monitoring device. One of ordinary skill in the art will appreciate that a large and growing number of connected monitoring devices are available, any of which may be used in various embodiments of the present invention to obtain physiological data.

The term “based on” is used to mean that a thing is determined at least in part by whatever it is described as being “based on”. If something is determined entirely by a thing, it will be described as being “based solely on” that thing. Where the term “based on” is used herein, embodiments are provided in which the thing is determined in part by whatever it is described as being based on and embodiments in which the thing is based solely on that thing. To “determine” something means to produce or generate, compute, establish, or specify that thing. For example, something may be “determined” by selecting it from a group of alternatives or options, by analyzing data (e.g., by applying an algorithm to the data) to generate a result.

The term “caregiver” refers to anyone who provides physical assistance with health care needs or activities of daily living to a patient outside a health care facility, typically on a reasonably frequent or regular basis. A caregiver may be a patient's family member, friend, or someone for whom caregiving is an occupation, job, or vocation. The term “remote caregiver” refers to anyone who is involved with or concerned about the patient's health but who typically does not provide the patient with physical assistance with health care needs or activities of daily living on a frequent basis. A remote caregiver may provide emotional support, e.g., through phone calls or other communication means, financial support, visits, or other forms of support. Remote caregivers may be, for example, children, siblings, or other family members living in different cities from the patient.

A “set” as used herein, is a collection of one or more things, which may be referred to as “elements” of the set. A “subset” of a set A is a collection consisting of any one or more elements of A, i.e., a subset of A does not include any elements that are not also in A. A subset of a set may include all the elements of the set. The elements of a set may be related to each other in some way, e.g., they may all be of a particular type, or they may all pertain to a particular patient.

The term “taper” is used to mean a process of gradual reduction. It should be understood that a “taper” may end in complete cessation of the thing reduced, or may result in a less than complete cessation. Similarly, a “taper” should be understood as including both reductions that proceed at a steady rate, and reductions whose rates may be modified (or even pause) during the course of an overall reduction.

The term “user” refers to an individual or entity that uses a system, apparatus, or product (e.g., a computer program product or database). An individual user may for example, be a health care provider, associated personnel, a patient, a patient's parent or legal guardian or a representative authorized by the patient to provide or access health data regarding the patient, a patient's caregiver, a researcher, an authorized employee or agent of an entity such as a health care institution, health care organization, payer, or pharmaceutical company, or anyone else who makes use of a system, apparatus, or product functionality or service. Users in any of the afore-mentioned categories may sometimes be referred to as “end users”. The term “user” also includes individuals who create, maintain, develop, update, administer, provide end user support, and/or otherwise support a system, apparatus, or product. Such users may be referred to as “support users”. Users may be authorized to use and/or modify a system, apparatus, and/or product to any extent compatible with their role(s). A user will typically have a user account or the right to use a user account. The user account allows a user to authenticate to a system, apparatus or product and be granted access to at least part of it or to one or more services provided by it. A user account is associated with a collection of data pertaining to the user and/or user account. User account data may comprise, for example, personal identifiers such as a user's name, password, biometric identifier (e.g., fingerprint, iris scan, photograph, characteristics of the individual's typing pattern), email address, business address, home address, etc. User account data also includes data indicating the extent to which the user is authorized to access, use and/or modify the system, apparatus, or product. In some embodiments, a system, apparatus, or product of the present invention comprises, creates, modifies, or accesses, a database that contains user account data.

### DETAILED DESCRIPTION

Described herein are systems and methods for supporting opioid tapering using an application, such as a mobile application, and complementary administration of cannabinoid compounds. Without wishing to be bound by any theory, aspects of this disclosure may be based on a recognition that one size fits all approaches are unlikely to be effective in a large number of cases, and so there may be benefits to technology that can dynamically adapt tapering for individual patients based on their individual circumstances and activities. While this disclosure includes various concrete embodiments, steps, compounds, systems and methods that could be used in the tapering of opioids, these examples, steps, compounds, systems and methods are intended to be exemplary only. Accordingly, the material set forth herein should be understood as being illustrative of the inventors' technology, but should not be treated as implying limits on the protection provided by this document or any document claiming the benefit of this document.

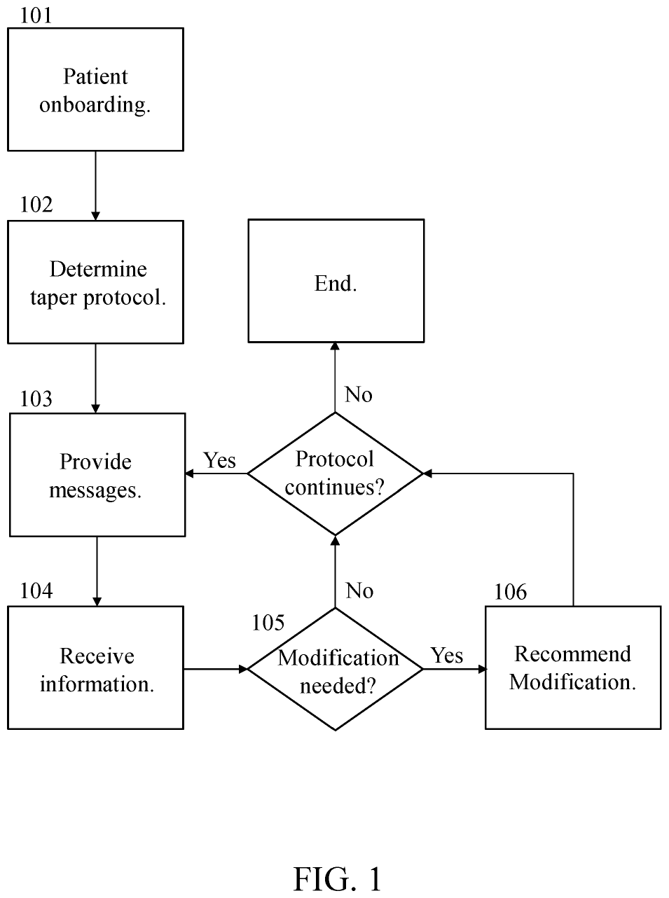

Turning now to the figures, FIG. 1 shows a potential approach to opioid tapering that may be implemented in some embodiments. As shown in FIG. 1, an opioid tapering process may begin with patient onboarding 101. This patient onboarding may include establishing data structures that would be used to store information about the patient, such as by establishing a profile for the patient with an entity maintaining a tapering application, linking the patient to a pre-existing profile maintained by such an entity, providing the patient with a copy of an application that could be resident on a computing device that was personal to him or her (e.g., a smartphone) and that would locally maintain a profile for the patient, or by performing other types of steps that may be appropriate for establishing such information storage structures in a particular embodiment. Patient onboarding 101 may also (or alternatively) include gathering one or more of the patient's (i) symptoms, (ii) pain level, (iii) medical history, (iv) sleep, (v) gender, (vii) height, (viii) weight, (ix) current opioid prescription(s). (x) current prescription(s) for non-opioid pain medications, (xi) current non-pain medicine prescriptions, (xii) current caregiver(s).

In some embodiments, other types of information may also be gathered as part of patient onboarding 101. For example, in instances where the disclosed technology is used in the context of gathering evidence that a cannabinoid (or other trial medication) is beneficial for opioid tapering, patient onboarding 101 may include gathering information for inclusions/exclusions for the trial. In embodiments where patient onboarding 101 includes gathering information on inclusions, the inclusions may include, for example, (i) that the patient has moderate to severe noncancer pain, (ii) that the patient is concerned about the side effects of pain medication (e.g., side effects of the opioid(s) that the patient is taking; adverse events in the form of undocumented therapeutic/pharmacological events that may be unforeseen or dangerous, or may be simply a reaction to new medication and/or combination of drug and opioid), (iii) that the patient is taking the opioid(s) that are the subject of the trial, and/or (iv) that the patient is concerned about addiction. In embodiments where patient onboarding 101 includes gathering information on exclusions, the exclusions may include, for example (i) having a history of an allergic reaction to cannabinoids (or other medication that is evaluated in the trial) or other ingredients in the medication, (ii) being a woman of childbearing age and not using reliable contraception, (iii) being pregnant or breastfeeding, (iv) being less than 18 years old, (v) having a history of schizophrenia or other psychotic disorders, (vi) having a serious heart disease, such as ischemic heart disease, irregular heartbeat or rhythm, poorly controlled blood pressure, or severe heart failure, (v) intends to start a family during the opioid taper, (vi) has a history of addiction or alcoholism, (vii) has terminal cancer or is receiving treatment for cancer. (viii) is taking rapid onset opioids for cancer breakthrough pain, and/or (ix) has used illicit drugs in the preceding year.

In different embodiments, information collection as part of patient onboarding 101 could be handled in different ways. For example, in some embodiments, patient onboarding 101 may be a collaborative process with the patient and a caregiver (e.g., a physician or physician office staff member) who could help the patient to enter the information using an interface (or set of interfaces) provided by an application implemented to facilitate the opioid taper. Similarly, in some embodiments, some (or all) of the information may be obtained through integration with an electronic healthcare record or pre-existing patient profile with the entity providing the application. In still other embodiments, some (or all) of the information may be obtained through integration between the application and one or more external monitoring devices (e.g., scales, sleep monitors, etc.). Combinations are also possible. For example, in some embodiments, an application used to assist in opioid tapering may retrieve some information from pre-existing electronic records, and then query the patient (or the patient's caregiver, or connected monitoring devices) for any additional information that may not be available from the existing electronic records. Accordingly, the description above of how information may be obtained as part of patient onboarding 101 should be understood as being illustrative only, and should not be treated as limiting.

Continuing with the discussion of FIG. 1, in that figure, after patient onboarding 101, a tapering protocol would be determined 102 for the patient. As with the patient onboarding 101, the determination 102 of a protocol could be performed in various different ways across embodiments. For example, in some embodiments, an application may be configured to determine 102 a protocol by automatically generating it for the patient. For instance, an application may be configured with a knowledge base (which may be local, or may be accessible over a network, depending on the embodiment) of different opioid products (e.g., morphine, hydrocodone, methadone, hydromorphone, and/or oxycodone) and associated recommended taper schedules for each (e.g., taper schedules specified by the Veteran's Administration, the American Pain Society, etc.) and may select one of the pre-configured schedules based on the opioid to be tapered for the particular patient. It is also possible that, in some such embodiments, a taper schedule may be determined based on more than just the opioid to be tapered. For example, in some embodiments, an application may query whether there is a timeline on which the taper is to be completed (e.g., because the taper is in preparation for surgery or other known event), in which case an appropriate taper (e.g., reduce by 2-5%/week, reduce by 5-10%/week, reduce by 10-20%/week, reduce by 20-50%/week) could be determined based on the specified timeline. It is also possible that, in some embodiments, a taper protocol may be determined by being provided by a physician for the individual patient. Combinations could also be made. For example, in some embodiments, the disclosed technology could be used to implement a system in which a physician would be provided with an interface including one or more protocols determined by the app, and then the physician would select and/or edit as appropriate for the particular patient. Accordingly, the discussion above of how a taper protocol could be determined 102 should be understood as being illustrative only, and should not be treated as limiting.

In a process such as shown in FIG. 1, after the taper protocol has been determined 102, the user could begin with the taper. During the course of the taper, an application implemented based on this disclosure could perform various acts to help improve the likelihood of success, such as providing 103 messages to the user. In various embodiments, these messages could be provided in various forms, such as alerts presented in the application itself, text messages sent through a separate text messaging channel, emails, audible alerts, spoken messages or any other type of notification that could be used to convey information to a user, e.g., on a screen, speaker or other output of a device he or she uses to execute and/or access the application. These messages could include messages such as medication reminders (e.g., if the user was instructed to take a medication at a certain time each day, then a reminder could be sent when that time was approaching), messages regarding non-medication activities (e.g., exercise) that may be beneficial, or messages of the day that would apprise the user of their current status in a pleasant motivational way and guide them to take whatever actions the app deemed necessary. Preferably, messages that would be provided 103 to the user would be personalized to help promote engagement and keep the user on track. In some embodiments, this type of dynamic message determination/personalization could be based on the user's status as set forth in table 1, below:

TABLE 1
Exemplary status based messages

| Status | Meaning | Potential Message |
|---|---|---|
| Getting | In an embodiment where the app | To best assist you, the app needs some |
| Started | was provided to the user before the | additional data. To provide this |
| | onboarding process was complete, | information, please click/touch here. |
| | this status could be used to indicate | |
| | that some additional information | |
| | needed to be provided. | |
| Evaluation | In an embodiment where an app is | We're gathering information to |
| | used to gather information about a | determine the best tapering protocol for |
| | patient's activities over time before | you. |
| | a taper is determined, this status | NOTE: this type of message may also |
| | could be used to indicate that the | include reminders or instructions for |
| | initial onboarding was complete | how the user could provide the |
| | and that the app was gathering | necessary information, such as |
| | information for use in determining | completing surveys or questionnaires, |
| | the taper. | providing consent to retrieve |
| | | information from art electronic medical |
| | | record, or connecting with an external |
| | | monitoring device. |
| On Track | The taper has been started and is | You're well on your way to wrapping |
| | being followed. | up with <insert opioid name>. At the |
| | | current rate of progress, you will be |
| | | completely off by <insert projected |
| | | taper end date> |
| | | NOTE: this type of message may be |
| | | accompanied by a grade or score to |
| | | encourage completeness, and may |
| | | provide a warning if it appears that the |
| | | patient is in danger of getting off track |
| | | (e.g., if the patient has taken his or her |
| | | medication at a time not indicated by a |
| | | predetermined schedule). |
| Waiting on | In embodiments where an | We're waiting on your revised |
| prescription | application is configured to | prescription. When it's approved, we'll |
| changes | recommend changes to a patient's | let you know. In the meantime, we'll |
| | prescription (e.g., change from 30 | continue with the protocol we're |
| | mg to 15 mg tablets so that opioid | already on. |
| | dosage can be reduced in a smaller | |
| | increment) this status could be used | |
| | to indicate that a recommendation | |
| | had been made to the patient's | |
| | physician, but that no confirmation | |
| | had been received that the | |
| | recommendation was accepted and | |
| | the modified prescription had been | |
| | filled. | |
| Off Track | This status may be used to indicate | Please contact your physician regarding |
| | that the tapering plan isn't being | continuation of your tapering protocol. |
| | followed to the degree necessary | |
| | for the app to continue. | |
| Physician | This status may be used when the | Please contact your physician regarding |
| Intervention | application (or a back end system | <detected scenario>. |
| | supporting the application) detects | |
| | a scenario that it cannot safely | |
| | handle. | |
| Support | This status may be used when the | Would you like to speak with a support |
| Intervention | application (or a back end system | representative? |
| | supporting the application) detects | NOTE: Preferably, embodiments |
| | that the patient is not properly using | which provide this type of message |
| | the app, or is having trouble using | would also include functionality that |
| | the app (e.g., because the patient | would automatically connect the |
| | repeatedly returns to a single input | patient to a support representative (e.g., |
| | screen without inputting any data or | through VoIP or chat interface) if the |
| | taking any other action). | patient indicated he or she did want to |
| | | talk to a representative. |
| Taper | This status may be used to indicate | You successfully completed the |
| Complete | that the tapering protocol has | tapering protocol. |
| | successfully completed. | |
| Clinical | This status may be used in | Your participation in the trial is on |
| Trial Hold | embodiments where an app is used | hold. The research coordinator will |
| | to gather information on efficacy of | contact you regarding your status |
| | a co-administered compound (e.g., | shortly. |
| | a cannabinoid) as part of a clinical | |
| | trial, but the patient is not correctly | |
| | participating in the clinical trial and | |
| | has been put on hold. | |

It should be understood that, in some embodiments, an application implemented based on this disclosure may also receive 104 information in addition to, or as an alternative to, providing 103 messages as described above. For example, in some embodiments, an application could be provided that included interfaces operable by a patient to record when, and how much, of the opioid being tapered is taken each time he or she takes it. Similar interfaces may also be used in some embodiments to record when the patient takes a cannabinoid or other compound being used as part of the opioid taper. Other information that may be received 104 from the patient in some embodiments includes periodic (e.g., weekly) pain and sleep surveys (e.g., PEG and PSQI surveys), the patient's activity levels (which may in some embodiments be used as a quality of life indicator), and any opioid withdrawal symptoms the patient may be experiencing.

In some embodiments which are configured to receive 104 information such as described above, that information may be used to determine 105 whether modifications are necessary to the patient's opioid taper, cannabinoids or other compounds the patient may be taking in conjunction with the taper, and/or the patient's opioid prescription. For example, in some embodiments, an application may, at the conclusion of each week during a taper, review the information received 104 from the patient to determine if the patient was having difficulty with the then active tapering protocol (e.g., because the patient was exhibiting withdrawal symptoms, because the patient was not reducing opioid use as expected or was becoming erratic with taking the opioid being tapered, because the patient was experiencing undesirably high levels of pain, etc.). Then, if the patient was having difficulty with the taper, the application could recommend 106 modifications to help facilitate the taper. These modifications could include slowing the pace of the taper (e.g., for the following week, instead of continuing to reduce the amount of opioid taken, continue with the amount of opioid taken in the previous week), increasing the use of (or, if such use had not yet begun, initiating) a cannabinoid or other complementary compound, or combinations of similar facilitative activities (e.g., pausing reduction in amount of opioid taken and increase use of cannabinoid). Alternatively, in some cases, the modification may be to modify the patient's opioid prescription, such as changing tablet size from 30 mg tablets to 15 mg tablets. Combinations of types of recommendations may also be possible in some embodiments. For example, in some cases, if the patient was having difficulty with the pace of a taper, an application may recommend increasing use a cannabinoid, decreasing the increment by which opioid use would be reduced in the following week, and reducing the size (dosage amount) of the opioid tablets the patient was prescribed to make it possible to decrease the increment by which the opioid use would be reduced.

In embodiments where an application will recommend 106 modifications, such recommendations may be made in a variety of manners. For example, in some embodiments an application may provide a recommendation through an interface or alert provided to the patient through app. In other embodiments, a recommendation may be made by sending a message to a patient's physician. In other embodiments, recommendations may be made through communication with both a patient and the patient's physician. For example, in a case where a recommendation is to change a patient's prescription, in some embodiments a message may be sent to the physician indicating the proposed change, and a message may be sent to the patient informing him or her to pick up the modified prescription if the physician indicates (e.g., through an interface provided by the application) that they are adopting the recommendation. Additional variations are also possible, and will be immediately apparent to those of ordinary skill in the art in light of this disclosure. Accordingly, the discussion above of how an embodiment may recommend 106 modifications should be understood as being illustrative only, and should not be treated as limiting.

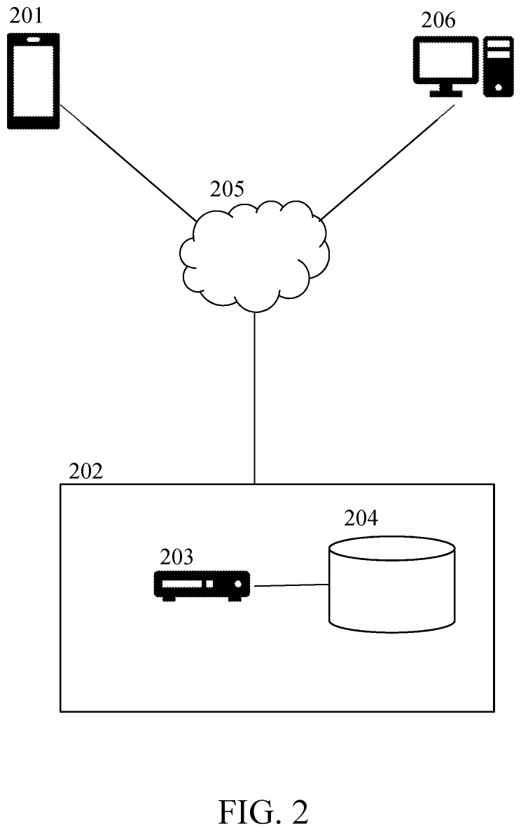

Turning now to FIG. 2, that figure illustrates an exemplary environment in which applications implemented based on this disclosure may be deployed. In the environment of FIG. 2, an application such as described previously would be installed on a patient's computing device 201. Such a computing device 201 may comprise any suitable type of computing device 201. For example, it may comprise a portable electronic device that a user may hold in his or her hand, such as a portable digital assistant (PDA), also referred to as a portable data assistant, a smartphone, a tablet computer, etc. The computing device may be a larger device, such as a laptop computer. As known in the art, PDAs are small, e.g., hand-held, computers, that are frequently used for tasks such as maintaining a calendar, list of contacts (e.g., email addresses), and other information. PDAs may contain application programs such as word processing programs, web browsers, PDF viewers, etc. Often, a computing device 201 such as illustrated in FIG. 2 may be implemented as a portable electronic device weighing under about 1-2 pounds, e.g., between about 3 ounces and about 1.5 pounds. For example, a smartphone or PDA may weigh between about 3 ounces and about 6 ounces and height and width dimensions in the range of less than about 7×5 inches and depth less than about 0.5-1.0 inch, though smaller or larger weight and/or dimensioned devices may be used. Exemplary computing devices may include, e.g., a PDA such as an iTouch (Apple, Inc.), a smartphone such as an iPhone (Apple, Inc.) or Galaxy phone (Samsung), or a tablet computer such as the iPad or iPad mini (Apple, Inc.). In some embodiments, a computing device 201 may be wearable, e.g., as a wristwatch, armband, etc. A computing device 201 may include components that may be found in such devices, e.g., control circuitry, storage/memory, input/output circuitry, communications circuitry, processing circuitry, etc. In some embodiments, one or more of such components of the device may be combined or omitted. In some embodiments, the computing device 201 may include other components such as, for example, a proximity sensor, a power supply such as a battery, a display, a positioning system, a camera, an accelerometer, an ambient light sensor, other sensors, an input mechanism, etc.) or multiple instances of one or more such components. In many embodiments, the portable electronic device may possess wireless connectivity. For example, the device may have Bluetooth, Wi-Fi wireless network connectivity, and/or the ability to connect to wireless Wide Area Networks, such as those provided by cellular telecommunications companies.

In addition to the computing device 201, the environment of FIG. 2 also includes a remote system 202 comprising a server 203 and a database 204 that is connected to the computing device 201 via a network 205. Additionally, the remote system 202 would also have a connection with a computer 206 that would be used by the physician overseeing the taper. As with the computing device 201, the remote system 202 and computer 206 may be implemented in a variety of manners, and the processing and data storage functions described above with respect to the process of FIG. 1 may be split among them in a variety of manners. For example, in some embodiments, an opioid taper implemented using a process such as shown in FIG. 1 could be implemented as a T-Plan of the type described in U.S. patent application Ser. No. 15/373,802 for Systems and Methods for Health Tracking and Management, the disclosure of which is hereby incorporated by reference in its entirety. In some embodiments, an opioid taper implemented as a process such as shown in FIG. 1 could be implemented as a set of rules for responding to events as described in U.S. patent application Ser. No. 15/202,223 for a Patient State Representation Architecture and Uses Thereof, the disclosure of which is hereby incorporated by reference in its entirety. In some embodiments, processing involved in implementing an opioid taper (e.g., analyzing information from a patient, determining whether changes are necessary) could be done on a system remote from the various users (e.g., remote system 202), with the users' devices (e.g., computing device 201 and computer 206) being used simply to provide interfaces for receiving data from, and communicating data to, the remote system. In some embodiments, a remote system could have a more limited role, such as a repository for information and hub for coordinating between devices, and the bulk of processing could be performed on the user's devices (e.g., computing device 201 and computer 206). Various other approaches, such as where processing is normally performed on a remote system, but would be performed on a user's device and subsequently synchronized in the event of connectivity issues, are also possible and will be immediately apparent to those of ordinary skill in the art in light of this disclosure. Accordingly, the above description of the environment of FIG. 2, along with various ways processing such as described in the context of FIG. 1 could be performed, should be understood as being illustrative only, and should not be treated as limiting.

Turning now to table 2, that table presents an exemplary tapering protocol for a 10%/week reduction in morphine use that could be programmed into or provided to an application such as described so that that application could assist with the taper (e.g., by providing determining and recommending potentially helpful doses of cannabinoids to take during the taper).

TABLE 2
Exemplary tapering protocol.

| | DAY 1 | | | | | | |
|---|---|---|---|---|---|---|---|
| WEEK 1 | (SUNDAY) | DAY 2 | DAY 3 | DAY 4 | DAY 5 | DAY 6 | DAY 7 |
| UPON WAKING | Take 1 60 mg and 1 15 mg pill or 5 15 mg pills with food (if you experience | | | | | | |
| | nausea) or without food as prescribed by your doctor (total 75 mg) | | | | | | |
| MIDDAY (8 hrs | Take 1 60 mg and 1 15 mg pill or 5 15 mg pills with or without food as prescribed | | | | | | |
| after waking) | by your doctor (total 75 mg) | | | | | | |
| BEFORE BED | Take 1 60 mg and 2 15 mg pills or 6 15 mg pills with or without food as prescribed | | | | | | |
| | by your doctor (total 90 mg) | | | | | | |
| | DAY 8 | | | | | | |
| WEEK 2 | (SUNDAY) | DAY 9 | DAY 10 | DAY 11 | DAY 12 | DAY 13 | DAY 14 |
| UPON WAKING | Take 1 60 mg and 1 15 mg pill or 5 15 mg pills with or without food as prescribed | | | | | | |
| | by your doctor (total 75 mg) | | | | | | |
| MIDDAY (8 hrs | Take 1 60 mg pill or 4 15 mg pills with or without food as prescribed by your | | | | | | |
| after waking) | doctor (total 60 mg) | | | | | | |
| BEFORE BED | Take 1 60 mg and 1 15 mg pill or 5 15 mg pills with or without food as prescribed | | | | | | |
| | by your doctor (total 75 mg) | | | | | | |
| | DAY 15 | | | | | | |
| WEEK 3 | (SUNDAY) | DAY 16 | DAY 17 | DAY 18 | DAY 19 | DAY 20 | DAY 21 |
| UPON WAKING | Take 1 60 mg pill or 4 15 mg pills with or without food as prescribed by your | | | | | | |
| | doctor (total 60 mg) | | | | | | |
| MIDDAY (8 hrs | Take 1 60 mg pill or 4 15 mg pills with or without food as prescribed by your | | | | | | |
| after waking) | doctor (total 60 mg) | | | | | | |
| BEFORE BED | Take 1 60 mg pill or 4 15 mg pills with or without food as prescribed by your | | | | | | |
| | doctor (total 60 mg) | | | | | | |
| | DAY 22 | | | | | | |
| WEEK 4 | (SUNDAY) | DAY 23 | DAY 24 | DAY 25 | DAY 26 | DAY 27 | DAY 28 |
| UPON WAKING | Take 1 60 mg pill or 4 15 mg pills with or without food as prescribed by your | | | | | | |
| | doctor (total 60 mg) | | | | | | |
| MIDDAY (8 hrs | Take 3 15 mg pills with or without food as prescribed by your doctor (total 45 mg) | | | | | | |
| after waking) | | | | | | | |
| BEFORE BED | Take 3 15 mg pills with or without food as prescribed by your doctor (total 45 mg) | | | | | | |
| | DAY 29 | | | | | | |
| WEEK 5 | (SUNDAY) | DAY 30 | DAY 31 | DAY 32 | DAY 33 | DAY 34 | DAY 35 |
| UPON WAKING | Take 3 15 mg pills with or without food as prescribed by your doctor (total 45 mg) | | | | | | |
| MIDDAY (8 hrs | Take 3 15 mg pills with or without food as prescribed by your doctor (total 45 mg) | | | | | | |
| after waking) | | | | | | | |
| BEFORE BED | Take 1 60 mg pill or 4 15 mg pills with or without food as prescribed by your | | | | | | |
| | doctor (total 60 mg) | | | | | | |
| | DAY 36 | | | | | | |
| WEEK 6 | (SUNDAY) | DAY 37 | DAY 38 | DAY 39 | DAY 40 | DAY 41 | DAY 42 |
| UPON WAKING | Take 3 15 mg pills with or without food as prescribed by your doctor (total 45 mg) | | | | | | |
| MIDDAY (8 hrs | Take 3 15 mg pills with or without food as prescribed by your doctor (total 45 mg) | | | | | | |
| after waking) | | | | | | | |
| BEFORE BED | Take 3 15 mg pills with or without food as prescribed by your doctor (total 45 mg) | | | | | | |
| | DAY 43 | | | | | | |
| WEEK 7 | (SUNDAY) | DAY 44 | DAY 45 | DAY 46 | DAY 47 | DAY 48 | DAY 49 |
| UPON WAKING | Take 3 15 mg pills with or without food as prescribed by your doctor (total 45 mg) | | | | | | |
| MIDDAY (8 hrs | Take 2 15 mg pills with or without food as prescribed by your doctor (total 30 mg) | | | | | | |
| after waking) | | | | | | | |
| BEFORE BED | Take 3 15 mg pills with or without food as prescribed by your doctor (total 45 mg) | | | | | | |
| | DAY 50 | | | | | | |
| WEEK 8 | (SUNDAY) | DAY 51 | DAY 52 | DAY 53 | DAY 54 | DAY 55 | DAY 56 |
| UPON WAKING | Take 2 15 mg pills with or without food as prescribed by your doctor (total 30 mg) | | | | | | |
| MIDDAY (8 hrs | Take 2 15 mg pills with or without food as prescribed by your doctor (total 30 mg) | | | | | | |
| after waking) | | | | | | | |
| BEFORE BED | Take 3 15 mg pills with or without food as prescribed by your doctor (total 45 mg) | | | | | | |
| | DAY 57 | | | | | | |
| WEEK 9 | (SUNDAY) | DAY 58 | DAY 59 | DAY 60 | DAY 61 | DAY 62 | DAY 63 |
| UPON WAKING | Take 2 15 mg pills with or without food as prescribed by your doctor (total 30 mg) | | | | | | |
| MIDDAY (8 hrs | Take 2 15 mg pills with or without food as prescribed by your doctor (total 30 mg) | | | | | | |
| after waking) | | | | | | | |
| BEFORE BED | Take 2 15 mg pills with or without food as prescribed by your doctor (total 30 mg) | | | | | | |
| | DAY 64 | | | | | | |
| WEEK 10 | (SUNDAY) | DAY 65 | DAY 66 | DAY 67 | DAY 68 | DAY 69 | DAY 70 |
| UPON WAKING | Take 2 15 mg pills with or without food as prescribed by your doctor (total 30 mg) | | | | | | |
| MIDDAY (8 hrs | Take 1 15 mg pill with or without food as prescribed by your doctor (total 15 mg) | | | | | | |
| after waking) | | | | | | | |
| BEFORE BED | Take 2 15 mg pills with or without food as prescribed by your doctor (total 30 mg) | | | | | | |
| | DAY 71 | | | | | | |
| WEEK 11 | (SUNDAY) | DAY 72 | DAY 73 | DAY 74 | DAY 75 | DAY 76 | DAY 77 |
| UPON WAKING | Take 1 15 mg pill with or without food as prescribed by your doctor (total 15 mg) | | | | | | |
| MIDDAY (8 hrs | Take 1 15 mg pill with or without food as prescribed by your doctor (total 15 mg) | | | | | | |
| after waking) | | | | | | | |
| BEFORE BED | Take 2 15 mg pills with or without food as prescribed by your doctor (total 30 mg) | | | | | | |
| | DAY 78 | | | | | | |
| WEEK 12 | (SUNDAY) | DAY 79 | DAY 80 | DAY 81 | DAY 82 | DAY 83 | DAY 84 |
| UPON WAKING | Take 1 15 mg pill with or without food as prescribed by your doctor (total 15 mg) | | | | | | |
| MIDDAY (8 hrs | Take 1 15 mg pill with or without food as prescribed by your doctor (total 15 mg) | | | | | | |
| after waking) | | | | | | | |
| BEFORE BED | Take 1 15 mg pill with or without food as prescribed by your doctor (total 15 mg) | | | | | | |
| | DAY 85 | | | | | | |
| WEEK 13 | (SUNDAY) | DAY 86 | DAY 87 | DAY 88 | DAY 89 | DAY 90 | DAY 91 |
| UPON WAKING | Take 1 15 mg pill with or without food as prescribed by your doctor (total 15 mg) | | | | | | |
| MIDDAY (8 hrs | Take 1 15 mg pill with or without food as prescribed by your doctor (total 15 mg) | | | | | | |
| after waking) | | | | | | | |
| | DAY 92 | | | | | | |
| WEEK 14 | (SUNDAY) | DAY 93 | DAY 94 | DAY 95 | DAY 96 | DAY 97 | DAY 98 |
| BEFORE BED | Take 1 15 mg pill with or without food as prescribed by your doctor (total 15 mg) | | | | | | |
| | DAY 99 | | | | | | |
| WEEK 15 | (SUNDAY) | | | | | | |
| | STOP | | | | | | |

In some embodiments that utilize a taper such as shown in table 2, the initial week may be used to acclimatize the patient to a cannabinoid or other therapeutic to be co-administered as part of the taper. This may be done by leaving the patient's opioid prescription untouched for an initial time period, e.g., the first week, and instead introducing the cannabinoid. The cannabinoid may be any suitable cannabinoid compound such as a single cannabinoid compound or a mixture of two or more different cannabinoid compounds. For example, in some embodiments the cannabinoid comprises of a mixture of major cannabinoids such as CBD (Cannabidiol) or Δ⁹-THC ((−)-trans-Δ⁹-tetrahydrocannabinol) and minor cannabinoids such as Cannabinol (CBN) or Cannabividarin (CBDV), or Cannabidivarinic acid (CBDVA), or Cannabigerolic acid (CBGA) or Cannabigerol (CBG) or Cannaidiolic acid (CBDA), or Cannabigerolic acid or Cannabichromene (CBC), or Tetrahydrocannabinolic Acid (THCA), or CBT (cannabacitran), or Cannabichromenic acid (CBCA) other minor cannabinoids. For example, a mixture of approximately 2 parts Δ⁹-THC to 1 part cannabidiol (CBD), approximately 1 parts Δ⁹-THC to 2 part CBD, approximately 1 part Δ⁹-THC to 1 part CBD to one part CBG, approximately 1 part Δ⁹-THC to 1 part CBD to one part CBN, or other suitable ratio or approximately 1 part CBD to one part CBDV to one part CBG or any other suitable ratio. As a further example, in some embodiments the cannabinoid comprises a mixture of approximately 3 parts tetrahydrocannabinol (THC) to 1 part cannabidiol (CBD), approximately 2 parts THC to 1 part CBD, approximately 1 part THC to 1 part CBD, approximately 1 part THC to 2 parts CBD, approximately 1 part THC to 3 parts CBD, or any other suitable ratio. This cannabinoid may be introduced in various amounts, such as starting with one pill/day and increasing over the course of the week to 3 pills/day, starting and staying at 3 pills/day for the entire week, starting at 3 pills/day and increasing to 4 pills/day, etc. The cannabinoid could also be administered in various forms. For example, as described in Bruni, N., et al., Molecules (2018) 23, 2478 (doi:10.3390/molecules23102478) https://www.mdpi.com/1420-3049/23/10/2478 and Urits, I., Borchart, M., Hasegawa, M. et al., Pain Ther (2019). https://doi.org/10.1007/s40122-019-0114-4, the entireties of each of which are hereby incorporated by reference, cannabinoids can be administered in the form of a spray providing approximately 2.7 mg of THC and 2.5 mg of CBD per spray (e.g., the product known as Nabiximols (Sativex®), GW Pharmaceuticals). In an embodiment utilizing such a spray, the spray administration may start with a low level (e.g., 1-4 sprays/day) and ramp up to a higher level (e.g., 8 sprays/day) by the end of the acclimatization period), may be at the higher level (e.g., 8 sprays/day) throughout the acclimatization period, or may begin at the higher level and be increased further (e.g., to 12 sprays/day) through the acclimatization period. In some embodiments, administration of cannabinoids in liquid form, such as a liquid formulation with a concentration of, e.g., between 10 mg/ml and 100 mg/ml of CBD that may be ramped up to a dosage of, for example, 0.2 mg/kg/day to 5.0 mg/kg/day during the acclimatization period, may also (or alternatively) be used. An exemplary product is Epidiolex® (GW Pharmaceuticals), a 100 mg/ml CBD oral solution. Other dosage forms, such as capsules, tablets, oil, topical creams, gums, gels, transdermal patches, etc. could be used as alternatives to, or in conjunction with, dosage forms such as described above. For example, in some embodiments an oral THC and CBD formulation known as PTL401, which utilizes a self-emulsifying oral drug delivery system in capsule format, may be used (Atsmon, J., et al. J Pharm Sci. 2018:107(5):1423-1429). Other formulations that may be used are described in Bruni, et al., Molecules 2018, 23, 2478, and references therein. In some embodiments, to maintain consistency of cannabinoid administration, an app such as described could provide the patient with reminders of when and how to take the cannabinoid (e.g., with meals, at a consistent time of day, etc.). The cannabinoid may be plant-derived (e.g., as an extract) or may be synthetically produced, or a combination thereof.

In some embodiments, an app such as described herein could also gather information that could be used to determine the impact of the cannabinoids and/or whether their use should be modified (or discontinued). For example, in some embodiments, after the first week of a taper such as shown in table 2, a questionnaire could be provided asking for information about side effects of the cannabinoid (e.g., dizziness, drowsiness, diarrhea) and, if those side effects were present, could decrease the patient's use of the cannabinoid or, in some instances, discontinue the patient's use of the cannabinoid (and, if the embodiment was used in the context of a trial of the cannabinoid as part of an opioid taper, could disqualify the patient from the trial). Thereafter, the patient could be asked questions about his/her breakthrough pain on a weekly basis throughout the taper. If the patient's breakthrough pain has gone down, then the administration of the cannabinoid could be continued at its then current level. Alternatively, if the breakthrough pain had not gone down, then the cannabinoid could be increased (e.g., from 3 pills/day to 4 pills/day, from 8 sprays/day to 10 sprays/day, etc.). In some embodiments, other factors could also be considered in determining whether the patient's cannabinoid dosage should be increased. For example, in some embodiments, the patient's opioid use (e.g., not tapering as expected) may also, or alternatively, be treated as triggers for increasing cannabinoid dosage.

Of course, it should be understood that the above description of including an acclimatization period in a cannabinoid facilitated taper is intended to be illustrative only, and should not be treated as limiting. For example, in some embodiments, cannabinoid usage may be initiated at the same time as the patient's opioid dosage was being reduced, or may be conditionally initiated with or without an acclimatization period depending, for example, on the patient's prior experience with cannabinoids. Further, it should be understood that the length of an acclimatization period may vary. For example, in some embodiments the acclimatization period may last for a week. In some embodiments the acclimatization period may be between 1 and 6 weeks long. Additionally, in some embodiments, the concept of an acclimatization period as an initial portion of a taper may be absent entirely. For example, in some embodiments, a dose of a cannabinoid may initially be determined for a patient (e.g., using methods such as described above in the context of an acclimatization period of an opioid taper), and then a taper may only begin after the cannabinoid dosage is determined. In this type of embodiment, the taper may be based on the determined cannabinoid dosage (e.g., a patient with a lower cannabinoid dosage may be recommended a slower taper, while a patient with a higher cannabinoid dosage may be recommended a faster taper), may be determined based on other factors (e.g., timing, as described previously in the context of determining a taper protocol 102 from a method as shown in FIG. 1), or may even be a self-taper (e.g., where a user is provided with a target and allowed to select or specify a rate at which to reach it, or where the user was encouraged to reduce opioid intake as far as possible at a rate they were comfortable with). In these types of embodiments, the actual performance of the taper could be facilitated by an application in the same manner described previously in the context of FIG. 1 (e.g., through information gathering, processing, messaging, etc.) regardless of the fact that, effectively, patient onboarding 101 comprised generating information (i.e., a cannabinoid dosage) rather than simply gathering information as described previously. Accordingly, the above discussion should not be treated as limiting the protection provided by this document, or by any related document, to only embodiments in which cannabinoid administration is initiated in an acclimatization period during which the patient's opioid prescription is kept constant.

In addition to being used to help with administration of cannabinoids, in some embodiments an app as described herein may also be used to recommend and assist with administration of other types of complementary medications. For example, in some embodiments, messages provided by an application such as described herein could assist with or recommend the use of anti-inflammatories, which may be an important part of a patient's pain management. For example, if a patient's initial opioid prescription was for a medication that contained both an opioid and an anti-inflammatory agent, the app might recommend addition of a separate anti-inflammatory to the patient's regimen as the amount of the medication containing an opioid and an anti-inflammatory is reduced over time. Other complementary medications may also be recommended in some embodiments. For example, table 3 provides medications that some embodiments may recommend based on a patient's symptoms.

TABLE 3
Example complementary medications that may be recommended and recommendation triggers
that may be used in some embodiments.

| Symptom | Medication | Dose | Duration | Notes |
|---|---|---|---|---|
| If you | Take Clonidine (First line) | 0.1 to 0.2 mg oral | Re-evaluate in 3 | |
| experience | | every 6 to 8 hours | to 7 days; taper to | |
| sweating, rapid | | hold dose if | stop; average | |
| heart rate, | | blood pressure | duration 15 days | |
| twitches/jerks | | <90/60 mmHg | | |
| | | (0.1 to 0.2 ma 2 | | |
| | | to 4 times daily is | | |
| | | commonly used | | |
| | | in the outpatient | | |
| | | setting) | | |
| | or take Baclofen | 5 mg 3 times | Re-evaluate in 3 | |
| | (alternative) | daily may | to 7 days; average | |
| | | increase to 40 mg | duration 15 days, | |
| | | total daily dose | may continue | |
| | | | after acute | |
| | | | withdrawal to | |
| | | | help decrease | |
| | | | cravings, should | |
| | | | be tapered when | |
| | | | it is discontinued | |
| | or take Gabapentin | 100 to 300 mg | | Can help reduce |
| | (alternative) | and titrate to 1800 | | withdrawal |
| | | to 2100 mg | | symptoms and |
| | | divided in 2 to 3 | | help with pain, |
| | | daily doses | | anxiety, and |
| | | | | sleep |
| If you | take Hydroxyzine | 25 to 50 mg three | | |
| experience | | times a day as | | |
| anxiety, | | needed | | |
| feelings of | or take Diphenhydramine | 25 mg every 6 | | but not if you |
| general unease | | hours as needed | | are 65 or older |
| (dysphoria), | | | | |
| watering eyes | | | | |
| (lacrimation), or | | | | |
| a runny nose | | | | |
| (rhinorrhea) | | | | |
| If you | take NSAIDs like | Naproxen 375 to | caution in patients | |
| experience | naproxen or ibuprofen | 500 mg twice | with risk GI | |
| muscle aches | | daily or ibuprofen | bleed, renal | |
| (Myalgias) | | 400 to 600 mg | compromise, | |
| | | four times daily | cardiac disease | |
| | or take Acetaminophen | 650 mg every 6 | | |
| | | hours as needed | | |
| | Or use topical medications | As needed | | |
| | (menthol/methylsalicylate | | | |
| | cream, lidocaine | | | |
| | cream/ointment) | | | |
| If you have | take Trazodone | 25 to 300 mg | | |
| trouble sleeping | | orally at bedtime | | |
| If you | take Prochlorperazine | 5 to 10 mg every | | |
| experience | | 4 hours as needed | | |
| nausea or an | or take Promethazine | 25 mg orally or | | |
| upset stomach | | rectally every 6 | | |
| | | hours as needed | | |
| | or take Ondansetron | 4 mg every 6 | | |
| | | hours as needed | | |
| If you | take Dicyclomine | 20 mg every 6 to | | |
| experience | | 8 hours as needed | | |
| abdominal | | | | |
| cramping | | | | |
| If you | take Loperamide | 4 mg orally | | |
| experience | | initially, then 2 | | |
| diarrhea | | mg with each | | |
| | | loose stool, not to | | |
| | | exceed 16 mg | | |
| | | daily | | |
| | or take Bismuth | 524 mg every 0.5 | | |
| | subsalicylate | to 1 hour orally, | | |
| | | not to exceed | | |
| | | 4192 mg/day | | |

It should be understood that the description above is intended to be illustrative only, and that variations, such as other functionality that may be provided by an app may also or alternatively be included. For example, in some embodiments, an application may provide a physician with a dashboard that allows him or her to manage and view the status of all of his or her patients who are using the application for opioid tapering. It may also provide physicians with alerts for changes in patient status (e.g., changes in status as described in table 1), and/or easy ways for the physician to review the files of patients where the application has recommended some kind of interaction between the physician and patient. Additionally, in some embodiments, information provided to an application may be used to help maintain healthcare records for the patient, such as in the context of a Revon system as described in the context of the applications incorporated herein. Further, in some embodiments, an application such as described herein may also include prescription management features, such as tracking the amount of a cannabinoid (or other medication) the patient is taking, comparing that against the amount prescribed for him or her, and providing notifications to the patient (and or his or her physician) when the prescription was close to running out. In some embodiments which include this type of prescription management functionality, a notification that could be provided when a prescription was close to running out may be presented in a form that would easily allow for a refill to be ordered (e.g., a notification in the form of an alert with a “Request Refill” button the patient could activate), thereby avoiding a situation where the patient would run out of a medication being used in the taper.

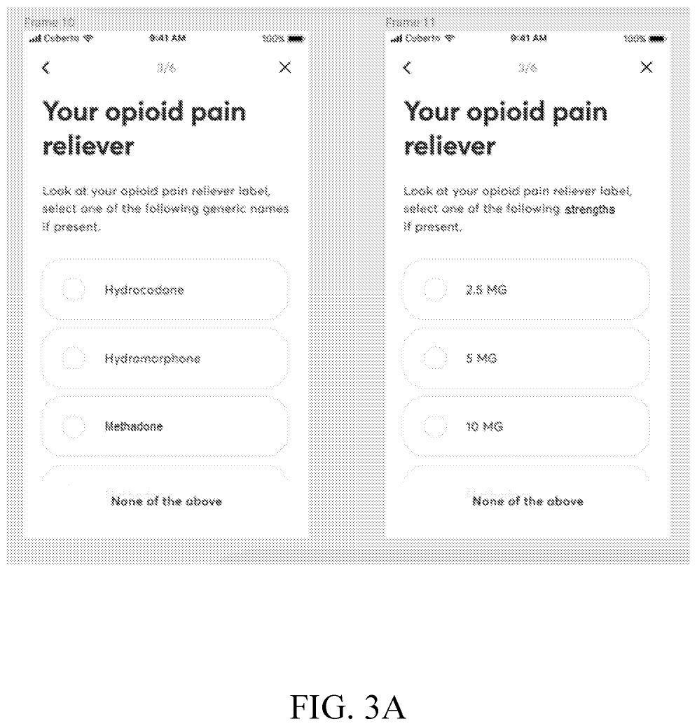

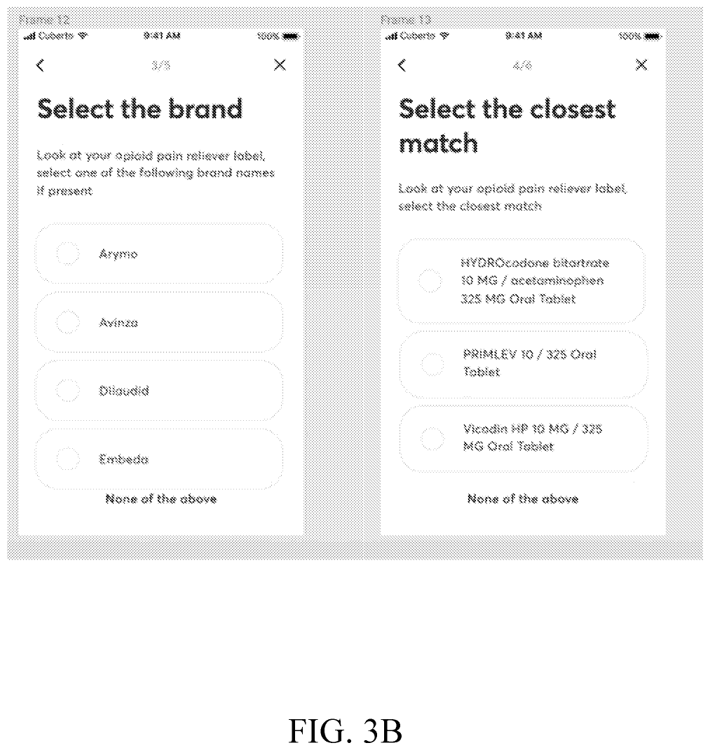

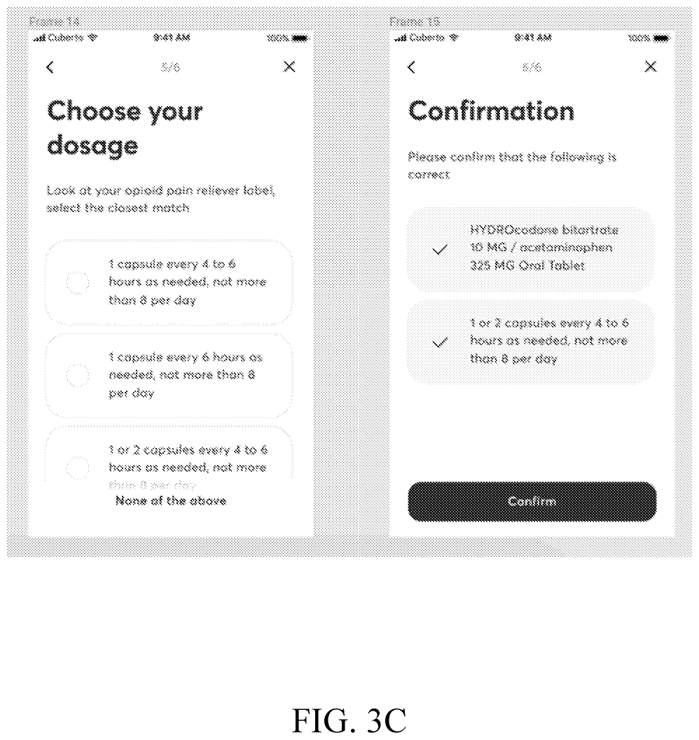

As another example of a type of additional functionality that may be included in some embodiments, in some cases an application implemented based on this disclosure may include features that could assist with specifying and understanding information regarding an opioid taper. For example, in some cases, an application may be configured with a knowledge base (e.g., stored in a remote system database 204, or on a user's computing device 201, in a publicly accessible database, or in a proprietary database maintained by a third party to which an entity providing an application as described herein has obtained access, etc.) that would include information needed to convert between brand names of medicines a patient is taking and the type and amount of opioid (and anti-inflammatory agent, if the product contains both an opioid and an anti-inflammatory) included in that product. This could be used, in some cases, to present interfaces in which a patient could specify the brand names and amounts of the medicine he or she was prescribed and/or was taking, and then automatically convert that information into substance and dosage information that could be used to determine and/or assist with a taper as described previously. For example, a patient might enter a product brand name and be presented with a list of various strengths (e.g., number of milligrams of opioid compound) and dosage formats (e.g., tablets, capsules) in which that product is available. The patient might then select his or her prescription from among the options. Examples of interfaces that may be presented to a patient for this purpose in some embodiments are provided in FIGS. 3A-3C. Similarly, information presented to a patient (e.g., a recommendation to take a particular amount of the tapered opioid at a particular time) could be converted to brand name information to be more easily understandable by the patient who would have to use that information. Information that could be included in a knowledge base to support this type of conversion could be of the type set forth below in table 4, and may be obtained from various sources, such as the Prescriber's Digital Reference, available at www.pdr.net. In some embodiments, prescription information might be entered by scanning a barcode on the drug product label. The patient may be asked to confirm that the prescription information obtained by scanning the barcode is correct.

TABLE 4
Exemplary brand, opioid and dosage conversion information.

| Brand | | Strength/ | Anti-inflammatory | Usual Adult |
|---|---|---|---|---|
| name | Opioid | Form | agent (if present) | Dosage |
| Lortab | Hydrocodone | 5 mg/ | 325 mg | 1 or 2 tablets |
| | | tablets | acetaminophen | every 4 to 6 |
| | | | | hours as needed |
| | | | | for pain. Total |
| | | | | daily dose should |
| | | | | not exceed 12 |
| | | | | tablets. |
| Lortab | Hydrocodone | 7.5 mg/ | 325 mg | 1 tablet every 4 |
| | | tablets | acetaminophen | to 6 hours as |
| | | | | needed for pain. |
| | | | | Total daily dose. |
| | | | | should not |
| | | | | exceed 6 tablets. |
| Lortab | Hydrocodone | 10 mg/ | 325 mg | 1 tablet every 4 |
| | | tablets | acetaminophen | to 6 hours as |
| | | | | needed for pain. |
| | | | | Total daily dose |
| | | | | should not |
| | | | | exceed 6 tablets. |
| Percocet | Oxycodone | 2.5 mg | 325 mg | 1 or 2 tablets |
| | | tablets | acetaminophen | every 6 hours as |
| | | | | needed for pain. |
| | | | | Total daily dose |
| | | | | should not |
| | | | | exceed 12 |
| | | | | tablets. |
| Percocet | Oxycodone | 5 mg | 325 mg | 1 tablet every 6 |
| | | tablets | acetaminophen | hours as needed |
| | | | | for pain. Total |
| | | | | daily dose should |
| | | | | not exceed 12 |
| | | | | tablets. |
| Percocet | Oxycodone | 7.5 mg | 325 mg | 1 tablet every 6 |
| | | tablets | acetaminophen | hours as needed |
| | | | | for pain. Total |
| | | | | daily dose should |
| | | | | not exceed 8 |
| | | | | tablets. |
| Percocet | Oxycodone | 10 mg | 325 mg | 1 tablet every 6 |
| | | tablets | acetaminophen | hours as needed |
| | | | | for pain. Total |
| | | | | daily dose should |
| | | | | not exceed 6 |
| | | | | tablets. |

Other types of variations, such including support for additional steps, may also be included in some embodiments. For example, in some embodiments, an opioid taper such as shown in FIG. 1 may have additional steps, such as an initial step of a physician prescribing the app for the patient who would undergo the taper, potentially in combination with a particular compound (e.g., cannabinoid) that would be taken as part of the taper. In some such embodiments, a cannabinoid may be prescribed together with the app, and the cannabinoid may have a label indicating that it is approved for use in conjunction with the app for purposes of facilitating opioid tapering. As another example, in some embodiments, an app such as described herein could continue to be used after a taper was finished. For example, in some embodiments, after a taper was complete (e.g., because the patient had reduced opioid use to zero, or to a maintenance dose in a case where the taper was designed to reduce but not eliminate the relevant opioid), an application implemented based on this disclosure could continue to provide reminders of any medications the patient continued to take, track the patient's status (e.g., pain level), and/or provide the patient's physician with the ability to monitor the patient's status to identify if there was a risk that the patient might return to using the opioid that had been tapered. Other variations are also possible, and will be immediately apparent to those of ordinary skill in the art. Accordingly, the description set forth above in the context of FIGS. 1 and 2 and tables 1-4 should be understood as being illustrative only and should not be treated as implying limits on the protection provided by this document or any related document.

It is expressly contemplated that each of the various aspects, embodiments, and features thereof described herein may be freely combined with any or all other aspects, embodiments, and features. The resulting aspects and embodiments (e.g., products and methods) are within the scope of the invention. It should be understood that headings herein are provided for purposes of convenience and do not imply any limitation on content included below such heading or the use of such content in combination with content included below other headings.

All articles, books, patent applications, patents, other publications, websites, and databases mentioned in this application are incorporated herein by reference. In the event of a conflict between the specification and any of the incorporated references the specification (including any amendments thereto) shall control. Unless otherwise indicated, art-accepted meanings of terms and abbreviations are used herein.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. It is to be understood that the invention encompasses all variations, combinations, and permutations in which one or more limitations, elements, clauses, descriptive terms, etc., from one or more of the listed claims is introduced into another claim. For example, any claim that is dependent on another claim may be modified to include one or more elements, limitations, clauses, or descriptive terms, found in any other claim that is dependent on the same base claim. Furthermore, where the claims recite a product (e.g., an apparatus or device or computer-readable medium), it is to be understood that methods of using the product according to any of the methods disclosed herein, and methods of making the product, are included within the scope of the invention, unless otherwise indicated or unless it would be evident to one of ordinary skill in the art that a contradiction or inconsistency would arise. A memory may comprise one or more non-transitory computer-readable media. In some embodiments, a memory may comprise at least a first medium and a second medium, wherein the first medium comprises a database and the second medium comprises the instructions. A database, or instructions, or both, may be stored on or divided among any number of computer-readable media, in various embodiments. An apparatus may comprise one or more processors. An apparatus may comprise one or more computer-readable media and one or more processors. A system may comprise an apparatus, which may itself comprise one or more systems or apparatuses. A claim expressed at least in part in terms a system may be expressed at least in part in terms of an apparatus (or apparatuses), or vice versa. Where a user or an act performed by a user are described, such user may, in at least some embodiments, be a designee of the user, and/or such act may be performed by a designee of the user, e.g., under direction of the user. Where elements are presented as lists, it is to be understood that each subgroup of the elements is also disclosed, and any element(s) may be removed from the group. The invention provides all such embodiments.

The terms “approximately” or “about” in reference to a number generally include numbers that fall within ±10%, in some embodiments ±5%, in some embodiments ±1%, in some embodiments 0.5% of the number unless otherwise stated or otherwise evident from the context (except where such number would impermissibly exceed 100% of a possible value). Where ranges are given, endpoints are included. Furthermore, it is to be understood that, unless otherwise indicated or otherwise evident from the context and understanding of one of ordinary skill in the art, values that are expressed as ranges may assume any specific value or subrange within the stated ranges in different embodiments of the invention, to the tenth of the unit of the lower limit of the range, unless the context clearly dictates otherwise. Any one or more embodiment(s), element(s), feature(s), aspect(s), component(s) etc., of the present invention may be explicitly excluded from any one or more of the claims.

1. A method of using an application in combination with a drug to implement an opioid taper for a patient comprising the application repeatedly performing a set of tapering steps comprising:
a) receiving information regarding progress of the patient on the opioid taper and using that information in evaluating progress of the patient on the opioid taper; and
b) providing one or more messages to the patient and information to the patient's physician based on the progress of the patient on the opioid taper, wherein at least one of the one or more messages provided to the patient comprises a recommended level for administration of the drug.

2. The method of claim 1, wherein the method further comprises administering the drug to the patient at the recommended level.

3. The method of claim 2, wherein the drug is a cannabinoid compound.

4. The method of claim 3, wherein the set of tapering steps comprises determining the recommended level for administration of the drug.

5. The method of claim 4, wherein:
a) the recommended level comprises a level prescribed by the patient's physician;
b) the one or more messages provided to the patient comprise a reminder to take the drug as prescribed by the patient's physician.

6. The method of claim 5, wherein:
a) the opioid taper comprises prescriptions by the patient's physician for an opioid and for the drug; and
b) the set of tapering steps comprises:
i) based on evaluation of progress of the patient on the opioid taper, determining whether to recommend modification to the prescriptions by the patient's physician for the opioid and/or for the drug;
ii) when a determination is made to recommend modification to the prescription by the patient's physician for the opioid and/or for the drug, sending messages to the patient and the patient's physician recommending the modification;
iii) when the patient's physician accepts a recommendation to modify the prescription for the drug, the recommended level comprises a modified level for the drug prescribed by the patient's physician.

7. The method of claim 6, wherein the application is configured to recommend modifying the prescription by the patient's physician for the opioid by changing the prescription for the opioid to a smaller unit size without changing formulation.

8. The method of claim 7, wherein:
a) the method comprises, during an acclimatization period, the application performing steps comprising:
i) providing one or more messages to the patient regarding continuing his or her opioid usage at a pre-taper level; and
ii) providing one or more messages to the patient regarding use of the drug;
b) the method comprises, after the acclimatization period but before performing the set of tapering steps, the application evaluating appropriateness of the drug for the patient; and
c) performance of the set of tapering steps is initiated based on determining that the drug is appropriate for the patient.

9. The method of claim 8, wherein:
a) the application is configured to, during and/or at the conclusion of the acclimatization period, gather drug side effect information for the patient; and
b) the application is configured to evaluate appropriateness of the drug for the patient based on the gathered drug side effect information.

10. The method of claim 9, wherein the information regarding progress of the patient on the opioid taper comprises:
a) outcome information regarding the opioid taper;
b) actual opioid usage; and
c) pain assessment data.

11. The method of claim 10, wherein:
a) the method comprises, after repeating the set of opioid tapering steps two or more times, ceasing the opioid taper with the patient on an opioid maintenance dosage; and
b) the opioid maintenance dosage is a lower dosage than the patient's pre-taper opioid usage level.

12. The method of claim 11, wherein:
a) the patient has a computing device which executes code for the application;
b) the patient's computing device is configured by the code for the application to communicate with a remote system over a network; and
c) evaluating progress of the patient on the opioid taper is performed by a server at the remote system.

13. The method of claim 12, wherein providing information to the patient's physician based on the progress of the patient on the opioid taper comprises providing the patient's physician an interface indicating:
a) amount of medication being taken by the patient on the opioid taper;
b) changes in status of the patient on the opioid taper.

14-15. (canceled)
